(19) United States
(12) Patent Application Publication
(10) Pub. No.: US 2023/0411002 A1
(43) Pub. Date: Dec. 21, 2023

(54) NIGHT LIGHT WITH EMBEDDED CELLULAR MODEM

(71) Applicant: VCCB Holdings, Inc., Wilmington, DE (US)

(72) Inventors: Joshua Seth Wachman, Newton, MA (US); David Loring Rose, Brookline, MA (US)

(21) Appl. No.: 18/327,863

(22) Filed: Jun. 1, 2023

Related U.S. Application Data
(63) Continuation of application No. 15/788,028, filed on Oct. 19, 2017.
Application No. 15/788,028 is a continuation-in-part of application No. 12/854,398, filed on Aug. 11, 2010.
Application No. 12/854,398 is a continuation-in-part of application No. 11/480,859, filed on Jul. 6, 2006, now abandoned.
Application No. 12/854,398 is a continuation-in-part of application No. 12/352,647, filed on Jan. 13, 2009, now abandoned.
Application No. 12/352,647 is a continuation-in-part of application No. 11/480,859, filed on Jul. 6, 2006, now abandoned.
Application No. 12/854,398 is a continuation-in-part of application No. 12/770,436, filed on Apr. 29, 2010.
Application No. 12/770,436 is a continuation-in-part of application No. 11/480,859, filed on Jul. 6, 2006, now abandoned.
Application No. 11/480,859 is a continuation-in-part of application No. 12/352,647, filed on Jan. 13, 2009, now abandoned.
(60) Provisional application No. 60/698,792, filed on Jul. 13, 2005.
(60) Provisional application No. 61/174,045, filed on Apr. 30, 2009.

(51) Int. Cl. G16H 40/67; H04M 1/21; G16H 40/63; G16H 80/00; G06Q 10/10; G16H 20/13

(52) U.S. Cl. CPC G16H 40/67; H04M 1/21; G16H 40/63; G16H 80/00; G06Q 10/10; G16H 20/13

(57) ABSTRACT

A device that includes a processor and memory; at least one long-range telemetry module, operatively connected to the processor, and providing connectivity between the device and remote networks, the at least one long-range telemetry module including at least one cellular radio; at least one short-range telemetry module, operatively connected to the processor, and providing connectivity between the device and local devices; and a display controlled by the processor, the display providing visual feedback.

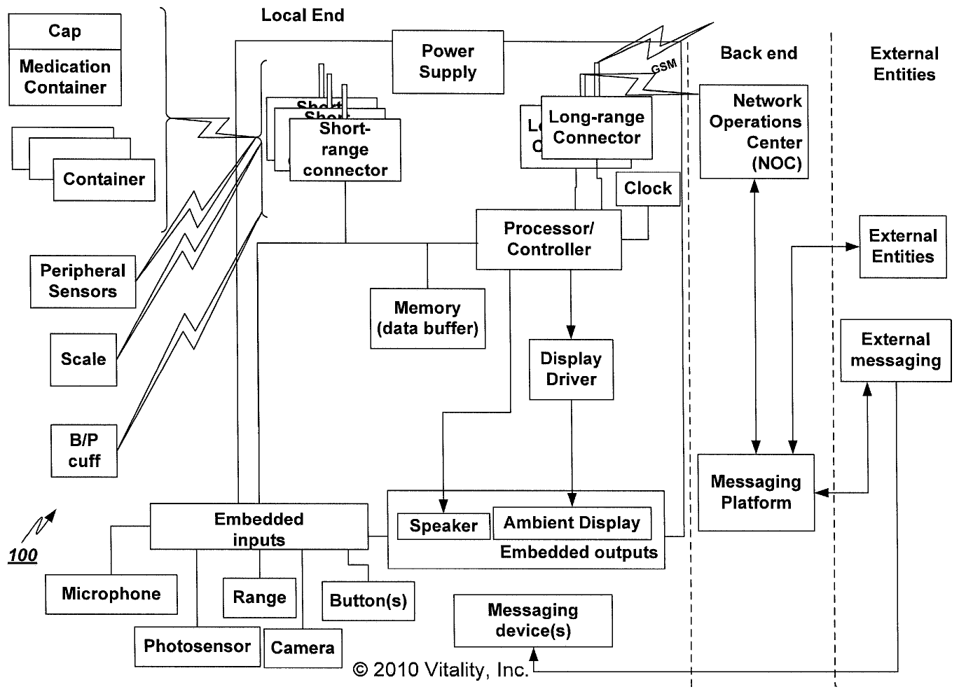

### RELATED APPLICATIONS

This application is related to the following United States patent applications, the entire contents of each of which are hereby fully incorporated herein by reference:
1. Appln. No. 61/174,045, filed Apr. 30, 2009, titled “Medicine bottle cap with an embedded curved display”.
2. application Ser. No. 12/770,436, filed Apr. 29, 2010, titled “Medicine Bottle Cap With Electronic Embedded Curved Display”.
3. application Ser. No. 11/480,859, filed Jul. 6, 2006, titled “Medication Compliance Systems, methods and devices with configurable and adaptable escalation engine.”
4. Appln. No. 60/698,792, entitled “Medication Compliance platform with intelligent networked pillbox, escalation engine and data signaling feedback loops,” filed Jul. 13, 2005.
5. application Ser. No. 12/352,647, filed Jan. 13, 2009, and titled “Medication Dispenser with Automatic Refill”.

This application claims priority from the following U.S. patent applications: application Ser. No. 15/788,028, filed Oct. 19, 2017; application Ser. No. 12/854,398, filed Aug. 11, 2010; application Ser. No. 12/770,436, filed Apr. 29, 2010; application Ser. No. 12/352,647, filed Jan. 13, 2009; and application Ser. No. 11/480,859, filed Jul. 6, 2006, each of which has been fully incorporated herein by reference, in its entirety, and for all purposes.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE DISCLOSURE

This invention relates to methods and devices for collecting data within the home environment including a night light for providing a visual alert.

### INTRODUCTION & BACKGROUND

Medication poor-adherence is a major problem in healthcare.

Physicians prescribe medications for a large class of diseases. Often, medications must typically be taken daily for the rest of the patient's life in order to sustain quality of life and reduce health risks. Classic examples of chronic diseases include hypertension, hypercholesterolemia and osteoporosis. With many such diseases, a patient feels no different, whether or not they take their medication. So, unlike brushing one's teeth or even exercising, there are no apparent short to medium term costs for poor-adherence. This presents a challenge even for those patients who want to comply, let alone those who need a helping hand.

Given a prescription medication taking regimen we shall define compliance as how well the patient sticks to the regimen and persistence as how long they stick to the regimen. Adherence is the product of compliance and adherence, and is the primary measure.

Various attempts have been made to try to increase and improve medication adherence. Almost all of these systems are basic reminder systems. For example, there are a large number of pillbox systems that marry alarm clocks to medication containers to remind patients when it is time to take their medication.

While various systems/devices are described here, we do not admit that any of them qualify as prior art to our invention.

There are some compliance intervention systems offered by health care providers designed to remind the patient and alert a remote caregiver so the caregiver can intervene in person. These include a sensor/reminders in the home, a network connection (typically dial-up) to a backend server and outbound messaging/reporting to a caregiver or even back to the patient. These systems, however, are focused on reminding only and while they may include a remote non-professional caregiver in the reminder loop, forgetfulness is only part of the problem.

Other systems try to help patients manage complex medicine regimens. For example, the MD2 device by Interactive Medical Developments of Aurora Healthcare is a coffee maker sized device that stores and dispenses pills like a common gum ball machine. The MD2 offers prerecorded audio messages to the patient and network connectivity back to a monitoring service. The MD2 is not designed to be portable, to be wirelessly connected to a network, to relay visual queues to another device resident in the home, or trigger escalating feedback to the patient. The focus on the MD2 is to arm disease management companies to assist patients on multiple medications and to help them effectively manage their regimen from home.

Remind Cap (of Remind Cap Pte. Ltd., Singapore) embeds a simple timer into the common cap that covers a medication bottle. It has an LCD display and audio alert which can be programmed for regular alarms.

MedPartner of Honeywell Hommed is a platform that helps patients manage complex medicine regimes. The MedPartner platform accommodates several pill bottles and alerts the patient when pills in their regimen needs be taken. The MedPartner system uses RFID technology to label the bottle and its location in an egg-crate like base station. It is networked to a healthcare provider's monitoring station (say in home care or nursing home environments).

SimPill of South Africa describes a pill bottle employing a GSM transmitter which reports to a cellular network whenever a pill is taken. They advertise that their system includes a “pill bottle which, when opened, delivers an SMS [short message service] text message to a central server. The SMS contains a unique pillbox ID number as well as some information about the battery status of the pillbox. Each SMS is time stamped. The central server receives the incoming SMS and, if it is within the time tolerances set for the pillbox sending the message is simply stored for statistical purposes. Should no message be received within the time tolerances then the server can be set to produce a number of responses (e.g., sending a text message reminder to the patient's handset, sending a text message prompt to a family member or community based care giver, prompting them to visit the patient to ascertain the cause of poor-adherence and provide assistance, sending a text message to a clinic based health professional or any other user determined response), or indeed escalate through these responses as time elapses with no incoming message in response to the previous outgoing message. Data on levels of compliance as measured by the device are stored for future analysis and use.” The SimPill device is ultimately another reminder system, based on its developer's theory (expounded on their website), that “[aJn important proportion of poor-adherence is caused by the patient simply forgetting to take their medication.” When a patient does not take her medication, SimPill reminds the patient and then, possibly, a caregiver. Like the other reminder/alarm systems, SimPill ignores the more complex nature of poor-adherence. GSM telemetry units are expensive and patients may be on several medications concurrently. By embedding a GSM module in each bottle, SimPill does not enable its solution to scale economically. Furthermore this approach requires pharmacies to dispense in a novel bottle which is challenges market adoption.

A category of medication compliance platforms has been developed specifically for the clinical trial market. In this market it is critically important to capture the dosing data of patients in order to measure their use and the medications efficacy during a clinical research trial. The price point of these devices is necessarily higher and they are built almost as a medical device to suit the stringent requirements of pharmaceutical manufacturers' clinical research requirements. For example, Informedix of Rockville, Maryland has a suite of products focused on compliance systems for the clinical trial market. Their Med-eMonitor is designed to be a clinical data capture diary and medication dispensing device in one. It has electronically monitored medication compartments and an instructional text screen. The device requires a cradle to upload the data and receive power. In the Med-eMonitor if the patient does not return the device to the base station there is no local or remote escalations to remind the patient to take their medication. The platform does not know if the device is even in the home. This suite of devices is designed for monolithic deployment-pharmaceutical companies deploy them in a research trial with a strict protocol that each subject patient must use to fulfill the requirements of the study.

Aardex (Aardex, Ltd. and associated company Aprex Corporation), a Swiss company offers a smart cap to fit standard vials for clinical trial dose recording. The AARDEX MEMS (Medication Event Monitoring System) product employs inductive and capacitive wireless uploading technologies that require close proximity to a networked base-station in the patient's home to upload to a personal computer or even a remotely networked back-end database. The device includes an LCD (liquid crystal display). In order to upload the data from the monitoring caps, a patient has to place it on back into a specially designed base station.

Some prior systems, e.g., as shown in U.S. Pat. No. 6,771,174, require a local computer system at each patient's home to monitor the patient's drug taking. The computer can contact a pharmacist or emergency services if the patient deviates from his or her model behavior. Such systems impose heavy cost requirements—a dedicated computer—at each patient's home. In addition, such systems cannot take advantage of information about other patients, in particular, how other patients have responded to various alert schemes. The inventors were the first to realize that it is desirable and useful to apply techniques to a patient that have been learned from other patients.

U.S. Pat. No. 7,081,807 to Lai discloses an electronic pill reminder device that that is retrofitted inside a regular conventional pill bottle cap—installed inside the conventional pill bottle between the bottle cap and the bottle container. When a user closes the pill bottle cap on the bottle container, an electronic timer, with a factory predetermined time interval, is activated. The timer generates alert signals to remind a user that a last pill has been taken and to remind the user to take his next dose.

The present invention improves on prior systems and overcomes their deficiencies.

### BRIEF DESCRIPTION OF THE DRAWINGS

The following description, given with respect to the attached drawing, may be better understood with reference to the non-limiting examples of the drawing, wherein: the drawing is an overview of a medication compliance system/framework.

### THE PRESENTLY PREFERRED EXEMPLARY EMBODIMENTS OVERVIEW

Well-established behavioral medicine research shows that poor-adherence with a medication regimen is fundamentally a behavioral psychology problem.

The inventors have realized that timely intervention(s) by machine or human may influence the patient and should increase medication adherence rates.

There are several reasons why patients may not comply with their medication regimens. No one reason or set of reasons may apply to all people. People are motivated in different ways and by different intrinsic or extrinsic factors, and it is an unknown and possibly a unique mix of factors that will motivate any particular individual to comply. The inventors have realized that any system for creating or supporting medication compliance will preferably be multi-faceted and be able to learn and adapt to each patient during their course of treatment.

Commonly acknowledged reasons for poor-adherence include the following:
Lack of doctor-patient accountability
High cost of medication
Lack of social support
Perceived difficulty or inconvenience of obtaining refills
Non-belief in the medications benefits
Poor understanding of how to take the medication
Forgetfulness
Unpleasant side effects

The inventors have realized that the most effective solution to address these common adherence barriers should address some or all of these factors.

### System Architecture

The drawing shows an exemplary medication compliance system/framework 100. For the purpose of this description, users of the system whose compliance is being monitored and affected are referred to as patients. The use of the word “patient” or “patients” in this description is not meant to limit the scope of the invention or to require any kind of doctor/patient relationship or any other kind of medical or legal relationship with the end users.

A compliance framework can be considered in three logical parts, namely the patients' homes (each a so-called “local end”), a back end, and a part corresponding to external entities that may be involved in the compliance system. The term “patient's home” is used herein to refer to the place (or places) at which a patient is expected to take his medication. It may include, e.g., the patient's home and/or place of work. The patient's home is sometimes referred to herein as the local end.

At a patient's home (or wherever they are supposed to take their medication), the patient is provided with a local system that includes a system manager, one or more feedback indicators and one or more connectors. The connectors allows the local end to connect with the rest of the system (e.g., the back end), and may be a modem, a network connection (wireless or wired) and the like. In presently preferred embodiments, as described in greater detail herein, some or all of these components may be integrated into a single device.

For example, the system manager, a feedback indicator, and the connector may be co-located and/or provided in a single device. Alternatively, e.g., the system manager and connector may be formed in a single device. If there is more than one feedback indicator, the system manager may be incorporated into one of them. In a presently preferred embodiment, as described in greater detail herein, the system manager, feedback indicator(s), and connector(s) are integrated in a single device, a “night light” indicator.

The patient's medication is provided in a container with a cap. The container may be a regular container or may be specifically adapted to operate with the cap, as described in the related applications.

Although only some medication containers are shown in the drawing (for the purposes of this description), it will be understood and appreciated that a patient may have a number of such containers for different medications. Additionally, a particular home (or location) may have medication containers for more than one patient.

A particular system may be open or closed. A closed system will only allow specific and dedicated caps to upload data through the network gateway. (The inventors characterize a closed system as a family of devices in which family members can talk to other family members.) An open system allows all caps to upload their data through the network gateway. (In an open system any member of one family can talk to members of another family.)

Thus, a particular patient may have more than one container (as shown in the drawing), each of which may have a cap and sensors as described above. Those skilled in the art will realize and understand, upon reading this description, that the number and type of containers will depend on the various medications that the particular patient is supposed to take, and that the containers need not all be the same size or type. E.g., some may contain pills; others may contain drops, blister packed medications, injectables, inhaleables, and so on.

The system assumes that if the medicine container has been opened and then closed, that the medication was actually taken and that the dosage was correct. The system is not designed to be fool proof, that is, the patient can defeat the system by opening and closing the cap and not taking out the correct dosage or any dosage and that is acceptable and intended design limitation. Preferably the number of pills has to be accounted for upon setup. This known number at the start is decremented by the dosage amount when the cap is opened, and is used to determine whether to initiate a refill. A local end may also include one or more peripheral sensors to measure and provide data such as the patient's weight, blood pressure (BP), pulse, etc. Peripheral measurements can be provided automatically to the system manager and, in some cases, may be requested by the system manager.

The various containers, sensors and feedback indicators may communicate with the system manager in any known way. The presently preferred implementation uses radio frequencies (ISM band) similar to that used in domestic garage door openers or key fob key-less entry systems. Other protocols such as Bluetooth®, ZigBee, Z-wave, 802.11, etc. may be used.

The system manager receives information from and about the sensors in its jurisdiction—the patient's home (in a closed system the system manager will only interact with known sensors). The system manager also communicates with the back end, e.g., via connector using, e.g., a network or a phone system. In some embodiments, the connector is a dedicated telephone dial-up, Ethernet or cellular modem called a network gateway. A network agnostic model may also be used where the network gateway has a plurality of embedded modems and, in the limiting case, the one with the lowest cost of connection, strongest signal or present availability (for instance) defines the connection used. The choice of connection is managed by the central processor or dedicated processor that is in receipt of decision making information from the network(s).

A network gateway is a device that connects the system manager to an external network via POTS (Plain Old Telephone Service) line modem, cellular, pager, 802.11 connections, or the like. In the POTS line modem version, the connector device may be embedded into a so-called “dongle”. In addition to the network connectivity, the dongle may communicate with the system manager over wireless, radio frequency communications.

In a presently preferred embodiment, the connector is a cellular modem that connects to the back end via a cellular telephone network.

The suite of devices described above communicates locally (in the home) and asynchronously from the virtual “backend” system components. Schematically these are local devices that communicate with the backend.

### Backend

The backend is a data service platform that manages individual patients' data. The structure and operation of the backend are described in detail in U.S. application Ser. No. 12/352,647, filed Jan. 13, 2009, and titled “Medication Dispenser with Automatic Refill,” (Atty. Docket No. 2694-0005), which has been incorporated herein by reference for all purposes.

### The Night Light Apparatus

Presently preferred embodiments of the night-light apparatus are described here. With reference to the drawing, the night-light apparatus consists of the following.

A power supply provides the apparatus with power through a direct connection to common wall socket. It should be apparent to those skilled in the art, upon reading this description, that batteries could replace this direct connection enabling the apparatus to be portable or that a wire could enable the device to sit remote from the wall connection with power either in a wall wart of embedded in the device and cord connecting to a power outlet. Those skilled in the art will also realize and understand that a combination of A/C power and battery power may be used, e.g., to provide an uninterrupted power supply in case of power outage.

A processor/controller, including a central processing unit (CPU) controls the apparatus. The CPU manages the network communications, display(s), alert(s), and all the subcomponents of the apparatus.

One or more long-range telemetry modules, including necessary antenna(e), support long range network connectivity. This includes an embedded cellular radio able to connect to the cellular network or a connection to any number of networks that enable the device to send and receive data, such as a pager network, WiFi, Wi-Max, or other wireless protocols that are to become more popular, etc.

One or more short-range (local) telemetry modules, including necessary antenna(e), provide short range network connectivity. This radio or radios may use any number of short range (i.e., ISM band) data protocols such as ANT, Bluetooth, Zigbee, RFID etc. connecting the apparatus to smart packaging (e.g., as described in the related applications), such as, e.g., GlowCaps™, blister packs, injectable syringes or injectable syringe cases, spirometers, scales, blood pressure cuffs, etc.

As the number of antennae increases concerns about cross talk becomes more acute and must be mitigated. To be effective, it is important that the antennae be protected from interference. Those skilled in the art of radio frequency and antenna design will recognize that proper electrical isolation (via shielding), judicial placement or temporal offset of transmission/reception signals, are popular techniques to enable the, at minimum two (local and long range), antennae signals from interfering with each other.

The device includes memory for buffering data if necessary. A so-called store-and-forward architecture enables the apparatus to collect data it senses in the home and store it until the time when the network is available. The buffer can also be used to receive remote data and store it until the nearby peripheral devices connects (comes in range). These data may be for programming new behaviors such as time of day, display information or parameters of measurement.

A crystal is used for precise time keeping which enables the device to be synchronized with universal time.

One or more status indicators, used, e.g., to provide an indication of the status of whether a medication needs to be taken, what the trend in blood pressure is, steps walked, or whether the person needs more medication. In some preferred embodiments, the status indicators include an embedded ambient display for indicating sensory status, connectivity status or analytics to passersby. In some embodiments, the display is an auxiliary ambient display, e.g., as made by Ambient Devices of Cambridge, Mass.

The apparatus may also, optionally, include one or more of the following:
an embedded graphical display for indicating sensory status, connectivity status or other information to passerby, for example weather, energy pricing, traffic congestion, or stock market trends may be used make the device relevant and interesting every day. The presently preferred embodiment has no alphanumeric display.
a button or set of buttons though in the preferred embodiment the apparatus is button free. Buttons could be used to trigger inbound or outbound calls. In bound calls may use, e.g., the push-to-refill system described in patent application Ser. No. 12/352,647, incorporated herein by reference. This functionality could also be embedded in the hub.
a speaker for broadcasting alerts or delivering medically relevant information to the home, or advertising information to the home from a supplier. The speaker and button above combo could enable the apparatus to function like a phone receiver. With the addition of a microphone it could become a full duplex telephone.
a microphone for enabling the hub to operate like a phone providing a real-time network voice connection to a remote location. The microphone could also be used to gauge presence of a person.
a voice radio (telephone) expanding the data services radio mentioned above.
a photo sensor to determine the ambient brightness near the hub enabling the displays to attenuate its brightness to be calibrated with the room.
a camera to collect photographs or video for capturing habits of daily living or detect which occupant in the home is present and when. This can be important to differentiate among inhabitants and assessing their patterns of daily living.
a motion or proximity sensor to detect activity and proximity of inhabitants. This may be used to determine whether a person is present, close enough to hear or see an alert or pausing in front of the hub. Some health care services can be offered on the condition of patient proximity or presence. For instance, this data can be used to tune the audio level of an alert or to not sound any alert if no one is nearby or deliver information if the person is standing in front of the hub with a product in range tacitly requesting information about that product.
temperature or smoke sensor for determining if fire is present or if environmental conditions are suitable.

### Overview & Operation

In operation, a user may look upon the apparatus specified above as a simple night light that plugs in under a kitchen counter or above a bathroom sink. While providing ambient information about the status of a key health metric like whether a medication needs to be taken, the apparatus offers highly relevant information at a glance. In a presently preferred embodiment the night light pulses amber whenever any medication needs to be taken and is otherwise steady blue.

As noted, the night light or similar device has one or more embedded cellular modems and one or more short range wireless transceivers. More generally it is an apparatus that communicates with other sensors local to the environment (using the short range wireless transceivers), and relays the sensory information from one location to another (via the one or more embedded cellular modems) to trigger various network services. The apparatus may also display information by illuminating and, in some cases, with textual and/or graphical display. The devices illumination source may change in color or pattern (or both) based on certain conditions.

The conditions triggering the display may be determined and set locally (e.g., based on received sensory information), or they may be determined and set remotely (and communicated to the device via the cellular modems).

The presently preferred application of this device is home health and wellness. The apparatus is capable of use for motivating behavior by subtly cueing passersby to the status of local sensors with one of several displays or alerts. The local sensors may include one or more of, e.g., wireless pill bottle caps, blood pressure cuffs, syringes or syringe cases, weight scales, blister packs, spirometers, pedometers, etc. Via its information relay feature, the apparatus can coordinate a variety of services from the home. In this manner, the apparatus may be considered a “home health gateway” facilitating the sharing of patient(s) health status to remote care-givers, service providers or facilities.

This so called “home health gateway” may assist home occupants with the management of their domestic supply of goods and services to keep the home operating efficiently. The supply chain may narrowly include the supply of medications or medical supplies (syringes, test strips, etc.), but more generally include any consumable staple of the home like food, paper products, printer supplies, milk, etc. The later becomes more valuable as so-called products with ‘smart packaging’ permeate the marketplace.

The term “home” is used here to specify a location, and is not intended to limit the scope of invention to use in any particular location or type of location.

So called “night lights” are common place in homes. By embedding a cellular radio and local wireless transceiver into a night light, the present apparatus augments existing functionality without requiring its owner to adopt any new behavior.

The functionality specified herein could be its own dedicated device or it could be embedded into some other device or appliance such as a refrigerator, phone, cell phone, light switch, picture frame, door knob, clock, lamp, or mirror, or some other ubiquitous tool of the modern home. The night light is presently the preferred form as the apparatus is primarily used for medication commonly stored in the kitchen or bathroom—a location in which night lights are most common. Night lights are common devices found in those rooms and may be located interchangeably in either. Furthermore both kitchens and bathrooms often have counter level wall sockets enabling the night light to be in view so when the device is more likely to be seen when it transitions to an alerting state.

If functionality of the apparatus is embedded into other tools, the resulting hybrid may channel other alerting mechanisms, consistent with the primary function of its host tool. For instance, a clock might offer a distinctive audio charm or a mirror may become semi-transparent revealing a display behind it that strategically overlays onto the viewer. By adopting a night light host for the apparatus the present embodiment provides a simple device easy to understand and use without requiring the user to adopt a new behavior, high cost of purchase or complicated installation.

As long range network availability and cost is variable, the apparatus may include logic to find the most robust and lowest cost network connection. This capability enables the apparatus to most reliably and most cost-effectively send and receive data. The simplest logic circuit (or firmware routine) would establish a cost associated with each potential network connection that is available and try the least expensive first, escalating as need be based on the criticality of the connection, network availability and cost. As the cost is also dynamic this metric would be received periodically from the network and used as an input to the decision logic in support of selecting the best connection before the next transmission.

### Personal Supply Chain

The night-light apparatus described here supports the smart pill bottle and related systems described in the related patents. The night-light apparatus specified herein enables a bottle or set of bottles to know the time of day it should alert, support a push-to-refill embedded button and alert the pharmacy or other caregivers when the bottle is depleted or near depleted so more medicine can be fulfilled.

While the system has been described with reference to a medication compliance system, it should be understood that the invention is applicable beyond that specific application. For example, the apparatus described herein enables and supports a scanner, camera, (or similar device) in the trash or recycling bin that can function with the expectation that any device that passes by the scanner is depleted and another unit needs to be ordered. The present device would place that order directly via traditional on-line commerce means or place the item into an on-line queue that the home-owner could affirm needs reordering. A similar device could be placed in or near the refrigerator to monitor presence of or stocks of food staples therein. Since most commercially available food has some form of bar code scan label or RFID tag on it or its package, this scanner need not be anything more elaborate than a bar code or RFID tag scanner. A camera may also send an image of the disposed item to the network to be interpreted by text/image recognition software or by a person. What would make it unique would be its ability to communicate wirelessly with the present apparatus and its capability to be embedded into the fridge, closet or recycling bin. When further connected to the purchasing behavior of the user the frequency of consumption, rate of expenditures or perhaps quality of ingredients in these expenditures can be tallied and displayed as another ambient indicator in the present invention.

As used herein, the term “medication” refers to any kind of medicine, prescription or otherwise. Further, the term “medication” includes medicine in any form, including, without limitation pills, salves, creams, powders, ointments, capsules, injectable medications, drops, vitamins and suppositories. The scope of this invention is not limited by the type, form or dosage of the medication.

Although the term “night light” is used herein to describe a preferred embodiment/implementation of the device, it should be understood that the device is not limited to nighttime use, and that the invention is not to be limited by the term “night light”.

Although aspects of this invention have been described with reference to a particular system, the present invention operates on any computer system and can be implemented in software, hardware or any combination thereof. When implemented fully or partially in software, the invention can reside, permanently or temporarily, on any memory or storage medium, including but not limited to a RAM, a ROM, a disk, an ASIC, a PROM and the like.

It is contemplated that various aspects of the present disclosure may be implemented by a mesh network.

While certain configurations of structures have been illustrated for the purposes of presenting the basic structures of the present invention, one of ordinary skill in the art will appreciate that other variations are possible which would still fall within the scope of the appended claims. While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1.-14. (canceled)

15. An alert display system comprising:
a processor;
a memory;
a first transceiver operatively connected to the processor, the first transceiver including at least one cellular radio, the first transceiver configured to communicate with a remote network;
a second transceiver operatively connected to the processor, the second transceiver configured to communicate with one or more sensing devices, the one or more sensing devices comprising a camera;
a display controlled by the processor and providing a visual alert as a function of signals received from the remote network or the one or more sensing devices;
a refill button configured to, in response to being actuated, cause the first transceiver to send an electronic message to a pharmacy or a caregiver that a prescription is nearly depleted or fully depleted; and
a trash or recycling can comprising the camera embedded in the trash or recycling can, wherein the camera is configured to capture one or more images of one or more items that pass by the camera,
wherein the first transceiver is configured to send the one or more images of the one or more items to the remote network,
wherein the processor is configured to:
determine whether the one or more items are depleted based on at least the one or more images, and
in response to determining that the one or more items is depleted, automatically place an order of the one or more items.

16. The system of claim 15, wherein the one or more sensing devices further comprises at least one of the following: a bar code scanner and an RFID tag.

17. The system of claim 15, wherein the visual alert provided by the display is associated with a color change of the illumination.

18. The system of claim 15, wherein the visual alert provided by the display is associated with a pattern change of the illumination.

19. The system of claim 15, further comprising a direct connection to a wall socket.

20. The system of claim 15, wherein the processor and the memory are disposed within a light switch operating as a night light.

21. The system of claim 15, wherein the processor and memory are disposed within a picture frame operating as a night light.

22. The system of claim 15, wherein the processor and memory are disposed within a mirror operating as the night light.

23. The system of claim 15, wherein the first transceiver is configured to find a network connection via the at least one cellular radio.

24. The system of claim 23, wherein the network connection is automatically selected by the first transceiver from multiple available connections based on an escalating need and dynamic cost metric.

25. The system of claim 15, wherein the at least one cellular radio supports bi-direction communications.

26. The system of claim 25, wherein the first transceiver is configured to operate as a duplex telephone.

27. The system of claim 15, wherein the visual alert reflects an escalation scheme operating as a function of a compliance regimen.

28. The system of claim 15, wherein the first transceiver is configured to communicate with the remote network responsive to a button free trigger.
